(19) United States
(12) Patent Application Publication
(10) Pub. No.: US 2023/0340462 A1
(43) Pub. Date: Oct. 26, 2023

(54) METHOD FOR PRODUCING DNA MOLECULES HAVING AN ADAPTOR SEQUENCE ADDED THERETO, AND USE THEREOF

(71) Applicant: RIKEN, Saitama (JP)

(72) Inventors: Yasunori ICHIHASHI, Saitama (JP); Tsuneo HAKOYAMA, Saitama (JP)

(21) Appl. No.: 17/799,177

(22) PCT Filed: Feb. 18, 2021
(86) PCT No.: PCT/JP2021/006057

§ 371 Date: Aug. 11, 2022

(30) Foreign Application Priority Data
Feb. 18, 2020 (JP) 2020-025576

(51) Int. Cl. C12N 15/10

(52) U.S. Cl. CPC C12N 15/1093

(57) ABSTRACT

The present invention provides a novel method for producing a DNA molecule having adapter sequences added thereto and a use thereof. An embodiment of the present invention provides a method for producing a DNA molecule having adapter sequences added thereto, the method including: a preparation step of preparing a double-stranded DNA in which a first DNA strand and a second DNA strand are at least partially hybridized; and an annealing step of annealing a partially double-stranded oligonucleotide adapter to a 3′ end of the first DNA strand of the double-stranded DNA, the partially double-stranded oligonucleotide adapter including a 3′ overhang which is a protruding end that is to be annealed to the 3′ end of the first DNA strand and that includes an oligonucleotide consisting of a random base sequence of at least 8 consecutive bases or a predetermined base sequence.

### TECHNICAL FIELD

The present invention relates to a method for producing a DNA molecule having adapter sequences added thereto, and a use thereof.

### BACKGROUND ART

With the spread of next-generation sequencers in recent years, it has become easier to read genetic information possessed by living organisms. Currently, the platform of the next-generation sequencer produced by Illumina, Inc. is widely used. For the sequencing with use of a next-generation sequencer, there is a necessity to prepare a DNA library sample in which sequences called adapters are added to both ends of a genomic DNA fragment to be analyzed, and a wide variety of kits for preparing the DNA library sample are commercially available. As these kits, for example, a genuine kit produced by Illumina, Inc., RThruPLEX (registered trademark) DNA-seq kit, and the like are well known.

However, these kits require, for example, a step of adding adapters with use of ligase, and are still expensive at prices of 6000 yen and up per sample. This imposes a heavy burden in handling a large number of specimens and is a major limitation of research.

Meanwhile, in Patent Literature 1 and Non-patent Literature 1, there have been reports on a method for preparing a library by producing a strand-specific cDNA from mRNA. In this method, cDNA is synthesized from mRNA, and an adapter sequence is inserted using a technique of inserting other sequences at the ends of a generated RNA-DNA duplex.

### CITATION LIST

### Patent Literature

### [Patent Literature 1]

Published Japanese Translation of PCT International Application Tokuhyo No. 2018-515081 (published on Jun. 14, 2018)

### Non-Patent Literature

### [Non-Patent Literature 1]

Townsley, B. T. et al. Frontiers in plant science, 6 (2015): 366.

### [Non-Patent Literature 2]

Yasunori Ichihashi and Atsushi Fukushima, “Frontiers of Transcriptomics in Plant Science”, BSJ-Review, 7:110 (2016)

### SUMMARY OF INVENTION

### Technical Problem

Unfortunately, the techniques described in Patent Literature 1 and Non-patent Literature 1 relate to RNA, and no study has been made on their application to DNA in Patent Literature 1 and Non-patent Literature 1.

Further, the existing commercial kits are expensive per sample. This imposes a heavy burden in handling a large number of specimens and is a major limitation of research.

As described above, further improvement in the preparation of DNA libraries has been desired.

An object of the present invention is to provide a novel method for producing a DNA molecule having adapter sequences added thereto and a use thereof.

### Solution to Problem

To solve the above problems, the present invention encompasses any one of the following aspects:
<1> A method for producing a DNA molecule having adapter sequences added thereto, the method including:
a preparation step of preparing a double-stranded DNA in which a first DNA strand and a second DNA strand are at least partially hybridized; and
an annealing step of annealing a partially double-stranded oligonucleotide adapter to a 3′ end of the first DNA strand of the double-stranded DNA,
the partially double-stranded oligonucleotide adapter including a protruding end (3′ overhang) that is to be annealed to the 3′ end of the first DNA strand and that includes an oligonucleotide consisting of a random base sequence of at least 8 consecutive bases or a predetermined base sequence.
<2> The method according to <1>, wherein a 5′ end of the first DNA strand, which constitutes the double-stranded DNA, includes a base sequence (second adapter sequence) that differs from each of base sequences of a double-stranded portion (first adapter sequence) of the partially double-stranded oligonucleotide adapter.
<3> The method according to <2>, wherein the preparation step includes preparing the double-stranded DNA by annealing, to a single-stranded DNA fragment corresponding to the second DNA strand, an adapter including: the oligonucleotide consisting of the random base sequence of the at least 8 consecutive bases or the predetermined base sequence; and the second adapter sequence located at a 5′ end of the oligonucleotide, and then extending a strand.
<4> The method according to <3>, wherein the adapter is annealed to the single-stranded DNA fragment corresponding to the second DNA strand at a temperature in a range of not lower than 30° C. and not higher than 50° C.
<5> The method according to any one of <1> to <4>, wherein the single-stranded DNA fragment corresponding to the second DNA strand is a collection of a plurality of DNA fragments obtained by fragmenting a genomic DNA and denaturing the fragmented genomic DNA into single-stranded DNAs.
<6> The method according to any one of <1> to <5>, wherein the method includes extending a strand from the protruding end included in the partially double-stranded oligonucleotide adapter to generate a third DNA strand complementary to the first DNA strand.
<7> The method according to any one of <1> to <6>, further including: an amplification step of amplifying the double-stranded DNA in which the first DNA strand and a third DNA strand complementary to the first DNA strand are hybridized.
<8> The method according to <7>, wherein a size of an obtained amplified fragment is in a range of not less than 300 bp and not more than 1000 bp.
<9> A DNA library for next-generation sequencer analysis obtained by the method according to <7> or <8>, the DNA library including a double-stranded DNA for analysis that is provided between at least a portion of the second adapter sequence and a sequence complementary to the second adapter sequence and at least a portion of a double-stranded portion (first adapter sequence) of the partially double-stranded oligonucleotide adapter.
<10> A kit for use in the method according to any one of <1> to <8>, the kit including at least one of the following (A) to (C):
(A) a partially double-stranded oligonucleotide adapter including a protruding end (3′ overhang) that is to be annealed to a 3′ end of a DNA strand and that includes an oligonucleotide consisting of a random base sequence of at least 8 consecutive bases or a predetermined base sequence;
(B) an adapter including: an oligonucleotide consisting of a random base sequence of at least 8 consecutive bases or a predetermined base sequence; and a second adapter sequence located at a 5′ end of the oligonucleotide; and
(C) a primer set consisting of a PCR primer that is to be annealed to a complementary sequence of the second adapter sequence and a PCR primer that is to be annealed to a strand (block strand), which does not have the protruding end, of the partially double-stranded oligonucleotide adapter.

### Advantageous Effects of Invention

According to the present invention, it is possible to prepare a DNA library at lower cost in a simpler manner.

### BRIEF DESCRIPTION OF DRAWINGS

FIG. 1 is a view illustrating an outline of a breath capture technique in accordance with an embodiment of the present invention.

FIG. 2 is a graph showing the ratio of sequenced genomic regions to a reference genome in each sample obtained in Example 1 and Reference Example.

FIG. 3 is a graph showing the ratio of read bases to a reference chromosome in each sample obtained in Example 1 and Reference Example.

FIG. 4 is a graph showing the mapping efficiency for the reference genome in each sample obtained in Example 1 and Reference Example.

FIG. 5 is a graph showing the ratio of sequenced genomic regions to the reference genome in each sample obtained in Example 5 in which 10 ng of genomic DNA of Drosophila melanogaster was used as an input.

### DESCRIPTION OF EMBODIMENTS

### Definitions of Terms and the Like

In the present specification, the term “polynucleotide” can be interpreted as meaning “nucleic acid” or “nucleic acid molecule”, and is intended to mean a polymer of nucleotides. Further, the term “base sequence” can be interpreted as meaning “nucleic acid sequence” or “nucleotide sequence”, and is intended to mean the sequence of deoxyribonucleic acid or ribonucleic acid, unless otherwise noted. Further, the polynucleotide may have a single-stranded structure or a double-stranded structure, and may be a sense strand or an antisense strand in the case of a single strand.

In the present specification, the term “gene” is interchangeable with “polynucleotide”, “nucleic acid”, or “nucleic acid molecule”. “Polynucleotide” means a polymer of nucleotides. Therefore, the term “gene” used in the present specification encompasses not only double-stranded DNA but also (i) single-stranded DNA, such as a sense strand and an antisense strand, by which double stranded DNA is constituted and (ii) RNA (such as mRNA).

In the present specification, the term “oligonucleotide” means a nucleotide polymer obtained by polymerization of a predetermined number of nucleotides. In the present specification, the length of the “oligonucleotide” is not limited. Note, however, that it is intended that the “oligonucleotide” is “polynucleotide” having a relatively short nucleotide chain.

In the present specification, the term “primer” refers to an oligonucleotide chain that is hybridized with a target or template nucleotide chain.

In the present specification, the term “DNA” encompass, for example, cDNA, genomic DNA, and the like each of which is obtained by cloning, a chemical synthesis technique, or a combination of cloning and a chemical synthesis technique. That is, the DNA can be (i) “genome” formed DNA that includes a non-coding sequence such as an intron in the form included in the genome of an animal or (ii) cDNA that can be obtained based on mRNA by use of a reverse transcriptase and a polymerase, that is, “transcription” formed DNA that includes no non-coding sequence such as an intron.

In the present specification, the term “RNA” refers to a nucleic acid having ribose sugar instead of deoxyribose sugar and generally having uracil instead of thymine as one of the pyrimidine bases.

All of the nucleobases in the present specification, including primers and oligonucleotides, may have one or more modifications known in the art (for example, chemical modifications and chemical substitutions, components of modified sugars, and chemiluminescent labels or fluorescent labels).

[1. DNA Molecule Production Method]

In an embodiment, the present invention provides a method for producing a DNA molecule having adapter sequences added thereto, the method including: a double-stranded DNA preparation step of preparing a double-stranded DNA in which a first DNA strand and a second DNA strand are at least partially hybridized; and an annealing step of annealing a partially double-stranded oligonucleotide adapter to a 3′ end of the first DNA strand of the double-stranded DNA, wherein the partially double-stranded oligonucleotide adapter includes a protruding end (3′ overhang) that is to be annealed to the 3′ end of the first DNA strand and that includes an oligonucleotide consisting of a random base sequence of at least 8 consecutive bases or a predetermined base sequence. The following description will discuss the individual steps of the present method in detail.

(1) Double-Stranded DNA Preparation Step

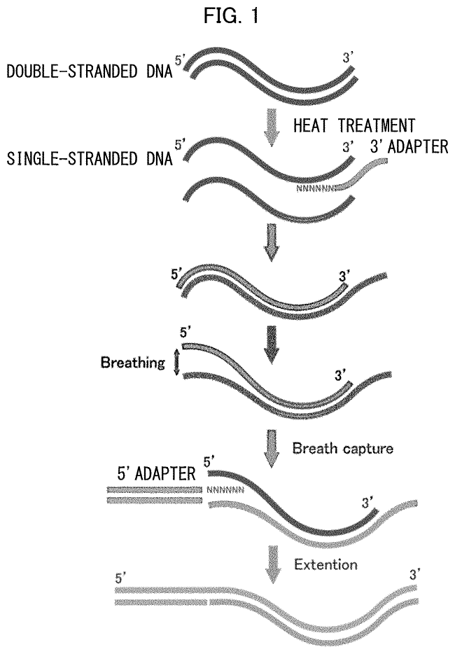

This step is a step of preparing the double-stranded DNA in which the first DNA strand and the second DNA strand are at least partially hybridized. In FIG. 1, this step is a step of preparing a double-stranded DNA illustrated in the third stage from the top of the sheet of FIG. 1. In the following description, when FIG. 1 is referenced, a lower-side strand of the double-stranded DNA illustrated in the third stage from the top of the sheet of FIG. 1 is referred to as a first DNA strand, and an upper-side strand thereof is referred to as a second DNA strand. In certain embodiments, in the double-stranded DNA prepared in the double-stranded DNA preparation step, 1) the first DNA strand and the second DNA strand are partially hybridized, 2) a 3′ end of the first DNA strand and a 5′ end of the second DNA strand form substantially flush ends, and 3) a 5′ end of the first DNA strand is not hybridized with the second DNA strand. Further, in a preferable embodiment, the 5′ end of the first DNA strand is configured by including a base sequence of known sequence (also referred to as a second adapter sequence and is distinguished from a first adapter sequence described later).

Note that, in the present specification, the term “adapter” or the term “adapter molecule” refers to an oligonucleotide that has a specific sequence and that is capable of being annealed to a target polynucleotide.

(1-1) DNA Fragmentation Step

In certain embodiments, the double-stranded DNA preparation step encompasses a DNA fragmentation step of fragmenting a DNA sample. Although not particularly limited, the DNA sample can be fragmented to a base length of preferably 300 bp to 1000 bp, more preferably 350 bp to 800 bp, and even more preferably 350 bp to 500 bp. In FIG. 1, a double-stranded DNA fragment illustrated in the first stage from the top of the sheet of FIG. 1 is an example of a DNA fragment obtained in the DNA fragmentation step.

This fragmentation step is carried out by, for example, heat-treating a genomic DNA. The conditions of the heat treatment are not particularly limited, but the heat treatment can be carried out by heating a solution containing extracted genomic DNA at, for example, 95° C. for about 45 minutes.

Examples of a solution for dissolving the extracted genomic DNA at the heating include 1 mM Tris (pH 7.5).

Other methods for the fragmentation include methods including an enzyme digestion treatment using, for example, a restriction enzyme, a shearing treatment, and an ultrasonic treatment.

DNA Sample

The DNA sample to be fragmented is not particularly limited, provided that it is a sample containing DNA. The DNA sample can be the one isolated from a sample derived from any living body including, for example, animals, plants, protists, yeasts, fungi, bacteria, and viruses (DNA sample isolation step). Examples of the plants include plants belonging to the families such as Gramineae and Brassicaceae, and examples of the animals include vertebrates such as mammals, birds, reptiles, and fish, and invertebrates such as insects, nematodes, and shellfish. As a method for isolating DNA, a known method can be used.

The DNA sample also includes a sample derived from an experimental plant such as Arabidopsis thaliana and a sample derived from an experimental animal such as Drosophila melanogaster. The DNA sample is not limited to a DNA sample derived from one kind of organism, and may be DNA samples derived from a plurality of kinds of organisms. Although not particularly limited, examples of the DNA samples derived from a plurality of kinds of organisms include samples for metagenomic analysis.

Examples of DNA contained in the DNA sample include genomic DNA and cDNA. Note that DNA includes wild-type DNA and DNA that has a single nucleotide polymorphism (SNP) or one or more mutations. Note that, although the genomic DNA is not particularly limited, substantially whole genomic DNA or a portion of genomic DNA collected by a method such as chromatin immunoprecipitation may be targeted as the genomic DNA.

(1-2) Step of Separating DNA Obtained in the DNA Fragmentation Step into Single-Stranded DNAs (Second DNA Strand Preparation Step)

In a case where the DNA fragment obtained in the DNA fragmentation step is a double-stranded DNA fragment, the double-stranded DNA fragment is separated into single-stranded DNAs. The separation of the double-stranded DNA fragment into single-stranded DNAs can be carried out by a known method such as heating at a predetermined temperature (what is called a “process for separating double-stranded DNA into single-stranded DNAs by thermal denaturation). In FIG. 1, this step is a step of separating a double-stranded DNA fragment into single-stranded DNAs, as illustrated in the second stage from the top of the sheet of FIG. 1.

That is, in certain embodiments, a single-stranded DNA fragment (corresponding to a second DNA strand of the double-stranded DNA that is prepared in the double-stranded DNA preparation step) is a collection of a plurality of single-stranded DNA fragments that are obtained by fragmenting a genomic DNA and denaturing the fragmented genomic DNA into single-stranded DNAs.

(1-3) Step of Preparing the First DNA Strand with Use of the Second DNA Strand

This step is a step of preparing the first DNA strand with use of the second DNA strand obtained in Section (1-2) above to prepare a double-stranded DNA composed of the first DNA strand and the second DNA strand. In FIG. 1, this step corresponds to a step illustrated in the second and third stages from the top of the sheet of FIG. 1.

In an embodiment, a single-stranded adapter (a 3′ adapter (or an adapter including the second adapter sequence)) including: an oligonucleotide consisting of a random base sequence of at least 8 consecutive bases or a predetermined base sequence; and a second adapter sequence located at a 5′ end of the oligonucleotide is annealed to a single-stranded DNA fragment (which serves as a template DNA fragment) corresponding to the second DNA strand (This corresponds to the second stage from the top of the sheet of FIG. 1.). After that, the first DNA strand complementary to the second DNA strand is extended by a primer extension reaction starting from a 3′ end (having an OH group) of the 3′ adapter. As a result, a double-stranded DNA, which is illustrated in the third stage from the top of the sheet of FIG. 1, is prepared in which the first DNA strand and the second DNA strand are at least partially hybridized. In this case, in the obtained double-stranded DNA, 1) the first DNA strand and the second DNA strand are hybridized such that a random oligonucleotide portion of the above-described 3′ adapter is a starting point, 2) a 3′ end of the first DNA strand and a 5′ end of the second DNA strand form substantially flush ends, and 3) the 5′ end (corresponding to the above-described second adapter sequence) of the first DNA strand is not hybridized with the second DNA strand. Note that, in the present specification, the expression “form substantially flush ends” encompasses not only a completely flush end but also a case where a deviation of 1 to several bases (for example, 5 bases, 4 bases, 3 bases, or 2 bases) occurs between the first DNA strand and the second DNA strand.

Note that, in the present specification, the case where two DNA strands are hybridized is not limited to a case where the respective base sequences of the two DNA strands are in a relationship such that they are completely complementary to each other in a region where hybridization can occur, unless otherwise limited. In the region where hybridization can occur, for example, one DNA strand may be an oligonucleotide having a sequence identity of 80% or more, preferably 85% or more, 86% or more, 87% or more, 88% or more, or 89% or more, more preferably 90% or more, 91% or more, 92% or more, 93% or more, or 94% or more, and even more preferably 95% or more, 96% or more, 97% or more, 98% or more, or 99% or more, with respect to the other DNA strand.

Similarly, in the present specification, the case where two DNA strands are complementary is not limited to a case where the respective base sequences of the two DNA strands are in a relationship such that they are completely complementary to each other in a region where hybridization can occur between the DNA strands, unless otherwise limited. In the region where hybridization can occur, for example, one DNA strand may be an oligonucleotide having a sequence identity of 80% or more, preferably 85% or more, 86% or more, 87% or more, 88% or more, or 89% or more, more preferably 90% or more, 91% or more, 92% or more, 93% or more, or 94% or more, and even more preferably 95% or more, 96% or more, 97% or more, 98% or more, or 99% or more, with respect to the other DNA strand.

The oligonucleotide portion constituting the 3′ adapter and consisting of a random base sequence or a predetermined base sequence need only be of a base sequence of at least 8 consecutive bases or a predetermined base sequence, but is preferably of a base sequence of not less than 6 and not more than 12 consecutive bases, and is more preferably of a base sequence of not less than 7 and not more than 9 consecutive bases. Note that, in the present specification, the “random (base sequence)” means encompassing all kinds of base sequences that can be interpreted as in the general definition of the word “random” (that is, in the case of a base sequence of n consecutive bases (where n is an integer of 2 or more), the random base sequence encompasses a base sequence of 4n kinds of bases).

Further, the “predetermined (base sequence)” means having a specific base sequence that is designed to anneal to, for example, a desired region at the 3′ end of the first DNA strand. By using such a base sequence, it is possible to generate a library of only a region having such a specific sequence.

Further, the second adapter sequence that constitutes the 3′ adapter can be selected so as to be compatible with a specific NGS platform. An example of the 3′adapter sequence is an oligonucleotide consisting of a base sequence shown in SEQ ID NO: 1.

Note that this step can be carried out in the presence of a DNA polymerase, the template DNA fragment (second DNA strand), and the 3′ adapter (functioning as a primer) under the conditions similar to the conditions of a primer extension reaction using a general random primer. Further, the description in the “Step (3) Extension step” section described later can also be referred to.

However, a temperature at which the 3′ adapter is annealed to the template DNA fragment affects the quality of a finally obtained library. For the purpose of stably obtaining a high-quality library, it is preferable that the 3′ adapter be annealed to the template DNA fragment at a temperature in a temperature range of not lower than 30° C. and not higher than 50° C. The temperature for the annealing is in a temperature range of more preferably not lower than 31° C., not lower than 35° C., not lower than 40° C., not lower than 42° C. and not higher than 50° C., not higher than 49° C., not higher than 48° C., or not higher than 47° C.

Note that the amount ratio between the 3′ adapter and the template DNA fragment (second DNA strand) is not particularly limited, but is preferably in a range of, for example, 1.4:1 to 69:1.

For the extension of the complementary strand of the template DNA fragment (that is, the formation of the strand), for example, a DNA-dependent DNA polymerase (for example, Klenow polymerase, Pol I DNA polymerase, etc.) can be used. For the strand extension, for example, a DNA polymerase and a deoxyribonucleotide (for example, dNTPs) are allowed to coexist in the presence of a suitable buffer solution, so that a strand extension reaction starting from the primer (here, the 3′ adapter) is carried out.

The DNA polymerase used for the strand extension has polymerase activity and 3′-5′ proofreading exonuclease activity, and may further include 5′-3′ exonuclease activity and/or terminal transferase activity. The DNA polymerase may be, for example, thermophilic DNA polymerase such as Taq DNA polymerase, Pfu DNA polymerase, Bst DNA polymerase, Tli DNA polymerase, Tfl DNA polymerase, Tth DNA polymerase, Vent DNA polymerase, SD DNA polymerase, and KOD DNA polymerase. Alternatively, the DNA polymerase may be, for example, mesophilic DNA polymerase such as Escherichia coli DNA polymerase I, Klenow fragment of Escherichia coli DNA polymerase I, phi29 DNA polymerase, T7 DNA polymerase, and T4 DNA polymerase. As an example, Ex Tag (Takara), KOD (Toyobo), Pfu (Agilent), PrimeSTAR HS (Takara), Q5 (NEB) Phusion High-Fidelity (NEB), Hifi (KAPA), Expand™ High Fidelity (Roche), or the like is used.

Further, in the extension reaction using the 3′ adapter, the temperature of the extension reaction is in a temperature range of not lower than 60° C. and not higher than 95° C., is preferably in a temperature range of not lower than 65° C. and not higher than 80° C., and is, for example, 72° C. or 74° C.

The rate of the extension reaction is not less than 0.01 kb/min and not more than 10 kb/min, is preferably not less than 0.1 kb/min and not more than 5 kb/min, and is, for example, 1 kb/min, 1.5 kb/min, or 2 kb/min.

In a case where MgCl₂ is used as an additive in the extension reaction solution, the concentration of MgCl₂ is not less than 0.01 mM and not more than 10 mM, is preferably not less than 0.1 mM and not more than 5 mM, and is, for example, 1 mM, 1.5 mM, or 2 mM.

Further, in a case where KCl is used as an additive in the extension reaction solution, the concentration of KCl is not less than 0.1 mM and not more than 1000 mM, is preferably not less than 1 mM and not more than 100 mM, and is, for example, 10 mM or 50 mM.

The concentration of dNTPs in the extension reaction solution is not less than 0.01 mM and not more than 10 mM, is preferably not less than 0.1 mM and not more than 1 mM, and is, for example, 0.2 mM, 0.25 mM, or 0.3 mM.

Further, if necessary, for example, 6 mM or 10 mM (NH₄)₂SO₄, 0.1% Triton X-100, and 0.001% or 0.1 mg/ml BSA are further added.

(2) Step of Annealing Partially Double-Stranded Oligonucleotide Adapter

The method in accordance with an embodiment of the present invention further includes an annealing step of annealing a partially double-stranded oligonucleotide adapter to the 3′ end of the first DNA strand of the double-stranded DNA obtained in the step (1) (double-stranded DNA preparation step). In FIG. 1, this step is a step illustrated in the fourth and fifth stages from the top of the sheet of FIG. 1. The first DNA strand is a lower-side strand, of the strands constituting the double-stranded DNA, drawn in FIG. 1.

Here, the “partially double-stranded oligonucleotide adapter” includes a protruding end (3′ overhang) that is to be annealed to the 3′ end of the first DNA strand described above and that includes an oligonucleotide consisting of a random base sequence of at least 8 consecutive bases or a predetermined base sequence (for example, corresponding to “NNNNNN” in a 5′ adapter in FIG. 1). Hereinafter, the partially double-stranded oligonucleotide adapter may also be referred to as a 5′ adapter.

The 5′ adapter includes a strand having an overhanging 3′ region (capture strand) and a shorter strand (block strand). That is, the 5′ adapter has a single-stranded portion and a double-stranded portion, and the block strand hybridizes with a portion of the capture strand. In the present specification, the double-stranded portion of the sequence of the 5′ adapter is also referred to as a first adapter sequence. In certain embodiments, the first adapter sequence (constituted by both strands) has a base sequence that is different from that of the second adapter sequence. For example, the first adapter sequence may have a sequence identity of 90% or less, 80% or less, 70% or less, or 60% or less with respect to the second adapter sequence. An example of the capture strand of the 5′ adapter is an oligonucleotide consisting of a base sequence shown in SEQ ID NO: 2. An example of the block strand of the 5′ adapter is an oligonucleotide consisting of a base sequence shown in SEQ ID NO: 3.

The case where the block strand and the capture strand are hybridized with each other is not limited to a case where the respective base sequences of these strands are in a relationship such that they are completely complementary to each other in a region where hybridization can occur. For example, the capture strand (except for the 3′ overhang) may be an oligonucleotide having a sequence identity of 80% or more, preferably 85% or more, 86% or more, 87% or more, 88% or more, or 89% or more, more preferably 90% or more, 91% or more, 92% or more, 93% or more, or 94% or more, and even more preferably 95% or more, 96% or more, 97% or more, 98% or more, or 99% or more, with respect to the block strand.

The first adapter sequence need only be, for example, a known base sequence of 8 consecutive bases, is preferably a base sequence of not less than 6 and not more than 12 consecutive bases, and is more preferably a base sequence of not less than 7 and not more than 9 consecutive bases. For example, the first adapter sequence can be selected so as to be compatible with a specific NGS platform. The specific NGS platform includes platforms commercialized by, for example, Illumina (registered trademark), Roche Diagnostics (registered trademark), Applied Biosystems (registered trademark), Pacific Biosciences (registered trademark), Thermo Fisher Scientific (registered trademark), Bio-Rad (registered trademark), and others. The same applies to the second adapter sequence. The first adapter sequence may further contain an index sequence or a barcode sequence which are designed to label either a target sample or a target sequence. In a certain example, these adapters can function sequencing adapters.

The oligonucleotide portion (which may be DNA or may be RNA) constituting the 5′ adapter and consisting of a random base sequence need only be of a base sequence of at least 8 consecutive bases or a predetermined base sequence, but is preferably of a base sequence of not less than 6 and not more than 12 consecutive bases, and is more preferably of a base sequence of not less than 7 and not more than 9 consecutive bases.

Note that the 5′ adapter can be prepared by, for example, hybridizing the above-described block strand and the above-described capture strand.

(2-1) Breathing Step

In certain embodiments, the step of annealing the 5′ adapter includes the step of breathing a double-stranded DNA to be annealed. The inventors of the present invention have previously developed the Breath Adapter Directional sequencing (BrADseq) library generation technique (Non-Patent Literature 1 and Patent Literature 1). This technique utilizes the fact that a double strand structure of a DNA/RNA complex involves a fluctuation (breathing), which is partial opening and closing, to specifically incorporate the adapter into a site at which the fluctuation (breathing) occurs.

In the double-stranded DNA obtained in the above-described step (1) (double-stranded DNA preparation step), in certain embodiments, the 3′ end of the first DNA strand (5′ end of the second DNA strand) is a substantially flush end, and the 5′ end of the first DNA strand is not hybridized with the second DNA strand. In certain embodiments, the breathing step is performed on such a double-stranded DNA having different forms at both ends.

In certain embodiments, the breathing step can be performed by allowing a solution containing the target double-stranded DNA and the 5′ adapter to stand at a temperature of, for example, not lower than 25° C.

(2-2) Step of Annealing the 5′ Adapter to the Double-Stranded DNA Having Undergone Breathing

In this step, the double-stranded DNA (in the fourth stage from the top of the sheet of FIG. 1) having undergone breathing in the above-described step (2-1) and the above-described 5′ adapter are allowed to coexist in a solution, so that the 3′ overhang, which is the protruding end of the partially double-stranded oligonucleotide adapter, is selectively annealed to the 3′ end (end having undergone breathing) of the first DNA strand of the double-stranded DNA.

This step can be performed after or concurrently with the above-described step (2-1). That is, the breathing of the double-stranded DNA and the annealing of the 5′ adapter can be performed in parallel.

Note that the condition under which the 5′ adapter is annealed to the double-stranded DNA is not particularly limited, but a temperature at which the 5′ adapter is annealed to the double-stranded DNA is preferably in a range of, for example, not lower than 20° C. and not higher than 30° C. The amount ratio between the 5′ adapter and the double-stranded DNA is also not particularly limited, but is preferably in a range of, for example, 14:1 to 713:1.

(3) Extension Step

The method in accordance with an embodiment encompasses, after or concurrently with the above-mentioned annealing step, forming a third DNA strand complementary to the first DNA strand by extending the strand from the protruding end (having an OH group) of the 5′ adapter.

In certain embodiments, the double-stranded DNA obtained by this step is configured by including: 1) a DNA duplex composed of the first DNA strand and the third DNA strand which is complementary to the first DNA strand; 2) one end constituted by a double-stranded portion of the 5′ adapter; and 3) the other end constituted by the 3′ adapter and a complementary sequence thereto. The ends in 2) and 3) are substantially flush ends.

For the strand extension (that is, the formation of the third DNA strand), for example, a DNA-dependent DNA polymerase (for example, Klenow polymerase, Pol I DNA polymerase, etc.) can be used. For the strand extension, for example, a DNA polymerase and a deoxyribonucleotide (for example, dNTPs) are allowed to coexist in the presence of a suitable buffer solution, so that a strand extension reaction starting from a primer (here, the protruding end of the 5′ adapter) is carried out.

The DNA polymerase used for the strand extension and/or the amplification has polymerase activity and 3′-5′ proofreading exonuclease activity, and may further include 5′-3′ exonuclease activity and/or terminal transferase activity. The DNA polymerase may be, for example, thermophilic DNA polymerase such as Taq DNA polymerase, Pfu DNA polymerase, Bst DNA polymerase, Tli DNA polymerase, Tfl DNA polymerase, Tth DNA polymerase, Vent DNA polymerase, SD DNA polymerase, and KOD DNA polymerase. Alternatively, the DNA polymerase may be, for example, mesophilic DNA polymerase such as Escherichia coli DNA polymerase I, Klenow fragment of Escherichia coli DNA polymerase I, phi29 DNA polymerase, T7 DNA polymerase, and T4 DNA polymerase. As an example, Ex Tag (Takara), KOD (Toyobo), Pfu (Agilent), PrimeSTAR HS (Takara), Q5 (NEB) Phusion High-Fidelity (NEB), Hifi (KAPA), Expand™ High Fidelity (Roche), or the like is used.

(4) Amplification Step

The method in accordance with an embodiment may include, after the above-mentioned step (3), an amplification step of amplifying the double-stranded DNA (double-stranded DNA in which the first DNA strand and the third DNA strand complementary to the first DNA strand are hybridized) obtained in the step (3).

Further, it is preferable that the concentration of the target double-stranded DNA and the addition of an adapter sequence be carried out by this amplification step.

As described above, in a typical example, a plurality of types of double-stranded DNAs to be amplified are each configured by including: 1) a DNA duplex composed of the first DNA strand and the third DNA strand which is complementary to the first DNA strand; 2) one end constituted by a double-stranded portion of the 5′ adapter; and 3) the other end constituted by the 3′ adapter and a complementary sequence thereto. The ends in 2) and 3) are substantially flush ends. That is, different sequences may be included in the two DNA strands in 1) above, but the portions in 2) and 3) above are common to the plurality of types of double-stranded DNAs.

In order to generate a plurality of amplified fragments to which the adapters are bound, in certain embodiments, the amplification step is performed by carrying out a PCR reaction with use of a PCR primer set having sequences corresponding to the 5′ adapter and the 3′ adapter (that is, sequences that are to be annealed to a part or whole of these adapters). In a more specific example, the amplification step is performed with use of a primer set consisting of a PCR primer that is to be annealed to the complementary strand of the 3′ adapter (second adapter sequence) and a PCR primer that is to be annealed to the block strand of the 5′ adapter (that is, a strand that does not have a protruding end). This produces a collection of DNA amplified fragments, as described later in the section “[2. DNA Library]”, having a common structure in which 1) a double-stranded DNA derived from a DNA sample is provided between 2) at least a portion (or whole) of the first adapter sequence (the double-stranded portion of the partially double-stranded oligonucleotide adapter) and 3) at least a portion (or whole) of a double-stranded portion constituted by the second adapter sequence and a sequence complementary to the second adapter sequence. Such a collection of DNA amplified fragments in which the adapter sequences are provided at both ends thereof can be used as, for example, a DNA library for next-generation sequencer analysis.

The amplification step performed by a PCR-based method will be described. In the presence of an appropriate PCR primer set, DNA polymerase (Poll) and dNTPs are first reacted in a suitable buffer solution. Each PCR cycle involves three common steps: denaturation, annealing, and extension. The temperature in the denaturation step is in a range of, for example, 90° C. to 100° C., and is 94° C. in one example. The duration of the denaturation step is in a range of, for example, 10 seconds to 10 minutes, and is 30 seconds in one example. The total number of PCR cycles is in a range of, for example, 10 to 50 cycles, is more preferably in a range of 16 to 21 cycles, but is not limited to these ranges. The temperature in the annealing step is determined according to the melting temperature of amplification primers. For example, the temperature in the annealing step is in a range of, for example, 50° C. to 70° C., and is 65° C. in one example. The duration of the annealing step may be in a range of, for example, 20 seconds to 4 minutes, and is 30 seconds in one example. The temperature in the extension step may be in a range of 68° C. to 75° C. The duration of the extension step is in a range of, for example, 10 seconds to 10 minutes, and is 30 seconds in one example. After the final extension step, an end extension step may be performed for, for example, 5 to 10 minutes, and for 7 minutes in one example.

The base length of the amplified fragments obtained through the above steps is not particularly limited, but is, for example, preferably 300 bp to 1000 bp, and more preferably 400 bp to 700 bp.

In a case where the amplified fragments obtained through the above steps are used for analysis by the next-generation sequencer, a sequence (called an insert) to be inserted in the next-generation sequencer is preferably not less than 300 bp.

Note that the amplification reaction is not limited to the PCR-based amplification method, and can include any DNA amplification reaction such as single primer isothermal amplification (SPIA), Ribo-SPIA, multiple displacement amplification (FDA), transcription amplification (TMA), nucleic acid sequence-based amplification (NASBA), strand displacement amplification (SDA), loop-mediated isothermal amplification (LAMP), helicase-dependent amplification (HAD), nicking enzyme amplification reaction (NEAR), and rolling circle amplification (RCA). Examples of the PCR-based amplification method include multiplex PCR, long-range PCR, routine PCR, high-speed PCR, hot-start PCR, touchdown PCR, and Nested PCR.

The extended and amplified DNAs may be size-selected and purified by size fractionation. Size fractionation may be performed by using SPRI beads (Ampure XP beads, Agencourt, Sera-Mag beads, etc.). Further, column chromatography (e.g., spin column), polyacrylamide gel electrophoresis, agarose gel electrophoresis, and the like can also be used.

(5) Sequence Determination Step

In some embodiments, the methods provided in the present specification further include the step of performing DNA sequencing of an amplification product obtained in the steps described above. Examples of DNA sequencing methods include automated sequencing using the Sanger method and sequencing using the next-generation sequencing (NGS) platform. Examples of the next-generation sequencing include, but not limited to, pyrosequencing, ion semiconductor sequencing, sequencing-by-synthesis with use of reversible dye-terminators, sequencing-by-ligation, sequencing-by-oligonucleotide probe ligation, and sequencing-by-synthesis with use of virtual terminators. As a next-generation sequencing system, for example, MiSeq (Illumina) can be used.

(6) Sequence Analysis Step

In certain embodiments, quantitative gene analysis further includes a sequence analysis step of performing analysis of a sequencing read.

Sequence analysis includes genomic equivalence analysis, single nucleotide variant (SNV) analysis, gene copy number variation (CNV) analysis, gene lesion detection, and sequence alignment. In a particular embodiment, such bioinformatics analysis is useful for quantification of the number of genomic equivalents analyzed in DNA clone libraries, detection of gene mutations and the like within a target locus, measurement of copy number variations within a target locus, and the like.

The methods described in the present specification are useful for preparation of DNA libraries used for a variety of purposes. The present methods can be combined with well-known sequencing techniques, especially high-throughput sequencing techniques.

[2. DNA Library]

Further, a DNA library for next-generation sequencer analysis obtained by performing the above-mentioned step (4) (amplification step) is also encompassed in the scope of the present invention. This DNA library is composed of a plurality of types of double-stranded DNAs for analysis. As described above, the plurality of types of double-stranded DNAs for analysis have a common structure in which 1) each double-stranded DNA for analysis is provided between 2) at least a portion (or whole) of the first adapter sequence (the double-stranded portion of the partially double-stranded oligonucleotide adapter) and 3) at least a portion (or whole) of a double-stranded portion constituted by the second adapter sequence and a sequence complementary to the second adapter sequence.

[3. Kit]

The present invention also provides a kit for use in the method, the kit including at least one of the following (A) to (C):
(A) a partially double-stranded oligonucleotide adapter including a protruding end (3′ overhang) that is to be annealed to a 3′ end of a DNA strand and that includes an oligonucleotide consisting of a random base sequence of at least 8 consecutive bases or a predetermined base sequence;
(B) an adapter including: an oligonucleotide consisting of a random base sequence of at least 8 consecutive bases or a predetermined base sequence; and a second adapter sequence located at a 5′ end of the oligonucleotide; and
(C) a primer set consisting of a PCR primer that is to be annealed to a complementary sequence of the second adapter sequence and a PCR primer that is to be annealed to a strand of a block strand (that is, a strand that does not have the protruding end) of the partially double-stranded oligonucleotide adapter.

The above (A) and (B) are materials for generating a sequence that serves as a template in a PCR afterward. The primer set in (C) is used to amplify a double-stranded DNA derived from a DNA sample having been generated as described above, together with the adapter sequences (A and B) at both ends. The primer set in (C) is based on the 5′ adapter and a strand extended from the 5′ adapter. In some embodiments, one primer of the primer set in (C) is complementary to the block strand of the partially double-stranded oligonucleotide adapter, and the other primer is complementary to the 3′ end of the extended strand.

The kit may contain a reagent necessary for preparing a DNA library. Examples of the reagent include suitable buffer solutions, suitable polymerases, DTT, dNTPs, sterile water, MgCl₂, DNA amplification primers, and reagents for purifying libraries. The kit can also include an instruction manual. The instruction manual may include instructions for carrying out the methods according to the embodiments described above.

As described above, in the present invention, it has been revealed for the first time that the breathing capture technique conventionally used for cDNA synthesis from mRNA can be applied to DNA. The method in accordance with an embodiment of the present invention makes it possible to easily prepare a DNA library in a short time. For example, in a case where the above-described kit is used, it is possible to generate the DNA library in about 1 to 2 hours.

As described above, the present invention proposes a DNA library preparation method that is lower in cost and is simpler and a DNA library prepared by using the DNA library preparation method. This method enables a DNA library to be prepared at low cost and to be of better quality than the conventional products.

The present invention is not limited to the embodiments, but can be altered by a skilled person in the art within the scope of the claims. The present invention also encompasses, in its technical scope, any embodiment derived by combining technical means disclosed in differing embodiments.

### EXAMPLES

### 1. Example 1: Generation of DNA Library

Experimental material: 1 ng, 10 ng, or 50 ng of genomic DNA of Arabidopsis thaliana (BioChain, D1634310-5) was used as an input.

Experimental method: The experiment was carried out by a procedure as indicated by the steps (a) to (d) below.

(Step (a): Acquisition of Fragmented dsDNA from Genomic DNA)

10 μl of 100 ng/μl Arabidopsis genomic DNA (Cosmo Bio Co., Ltd., D16343410-5) was taken, and 90 μl of 10 mM Tris was added. A solution thus obtained was dispensed by 20 μl each, and the dispensed solutions were heated at 95° C. for 45 minutes to fragment the DNA. AMPure XP beads (Beckman Coulter, A63880) 1.5 times as much as the volume of the solution were added, and the fragmented DNA was purified according to the specified manual. The fragmented DNA was eluted with 20 μl of water.

(Step (b): Denaturation from dsDNA to ssDNA and Priming of 3′ End of ssDNA)

For the dsDNA obtained in the step (a) above, denaturation to ssDNA and priming of a 3′ end of the ssDNA were carried out by a procedure as indicated by 1) to 8) below. For the fragmented DNA sample with each concentration, two different annealing temperatures of 35° C. and 45° C. were set.
1) The following materials were mixed.
5 μl of fragmented DNA (0.2 ng, 2 ng, or 10 ng/μl) 1 μl of 3-prime priming adapter (SEQ ID NO: 1: 5′-GTGACTGGAGTTCAGACGTGTGCTCTTCCGATCTNNNNNNNN-3′) (5 μM L-3ILL-N8.2)
1.5 μl of 10× Buffer (500 mM Tris-HCl (pH 7.5 at 25° C.), 100 mM MgCl₂, 10 mM DTT) (Takara Bio Inc., RR006)
1.2 μl of dNTPs
6.225 μl of H₂O
0.075 μl of Ex taq (Takara Bio Inc., RR006)
   15 μl in total
2) Incubation was carried out in a thermal cycler running the following program: 94° C. for 2 minutes; 35° C. or 45° C. for 10 minutes; 42° C. for 10 minutes; 72° C. for 5 minutes; and holding at 4° C.
3) 5 μl of 50 mM EDTA was added.
4) 30 μl of Ampure XP beads (Beckman Caulter) was added and mixed for size selection.
5) The resultant mixture was left to stand for 5 minutes.
6) The supernatant was discarded.
7) The remainder was washed twice with 200 μl of 80% EtOH.
8) The beads were dried.

(Step (c): Breath Capture of 5′ End of ssDNA (Breath Capturing))

Next, breath capture of a 5′ end of the ssDNA obtained in the step (b) above was carried out by a procedure as indicated by 1) to 8) below.

1) 4 μl of 10 μM 5-prime double stranded adapter oligo (5pSense8n (SEQ ID NO: 2: 5′-CCTACACGACGCTCTTCCGATCTNNNNNNNN-3′) and 5pAnti (SEQ ID NO: 3: 5′-AGATCGGAAGAGCGTCGTGTAGG-3′) was added.
2) The following mixtures were added.
1 μl of 10× Buffer (500 mM Tris-HCl (pH 7.5 at 25° C.), 100 mM MgCl₂, 10 mM DTT)
0.25 μl of 25 mM dNTPs
0.25 μl of DNA Pol I (Thermo Fisher Scientific, EP0041)
4.5 μl of H₂O
   6 μl in total
3) Incubation was carried out in a thermal cycler running the following program: 25° C. for 15 minutes.
4) The following mixtures were added.
10 μl of 50 mM EDTA
30 μl of ABR
   40 μl in total
4) The resultant mixture was left to stand for 5 minutes.
5) The supernatant was discarded.
6) The remainder was washed twice with 200 μl of 80% EtOH.
7) The beads were dried.
8) Elution was carried out with 30 μl of 10 mM Tris.

(Step (d): Concentration and Addition of Adapter Sequences)

Subsequently, the DNA eluted in the step (c) above was concentrated, and the adapter sequences were added by a procedure as indicated by 1) to 5) below.
1) The following PCR mixture was prepared:
2 μl of 10× Buffer (500 mM Tris-HCl (pH 7.5 at 25° C.), 100 mM MgCl₂, 10 mM DTT)
1.6 μl of dNTPs
1 μl of 2 μM PE1 (SEQ ID NO: 4: 5′-AATGATACGGCGACCACCGAGATCTACACTCTTTCCCTACACGA CGCTCTTCCGATCT-3′)
1 μl of 2 μM PE2 (SEQ ID NO: 5: 5′-CAAGCAGAAGACGGCATACGAGAT-index 8nt-GTGACTGGAGTTCAGACGTGTGCTCTTCCGAT-3′) (use a different index for each sample)
4.3 μl of H₂O
0.1 μl of Ex Taq
10 μl of breath captured DNA
20 μl in total
2) Incubation was carried out in a thermal cycler running the following program:
94° C. for 2 minutes; (21 cycles for 1 ng of input genomic DNA, 18 cycles for 10 ng, 16 cycles for 50 ng)×(94° C. for 30 seconds; 65° C. for 30 seconds; 72° C. for 30 seconds); 72° C. for 7 minutes; and holding at 4° C.
3) Ampure XP beads purification was carried out with use of 0.8× beads (washing was carried out twice).
4) Elution was carried out with 10 μl of 10 mM Tris.
5) Sequencing was carried out by MiSeq (Illumina, Inc., model number).

[Reference Example: Generation of DNA Library by Existing Technology]

Further, samples were prepared with use of 10 ng and 50 ng of input genomic DNA according to the respective standard protocols of Illumina, Inc. (TruSeq ChIP Sample Preparation Kit v2—Set A, IP-202-1012) and Takara Bio Inc. (SMARTer (registered trademark) ThruPLEX (registered trademark) DNA-seq 6S(12) Kit, R400523) as the conventional techniques.

As the experimental material, the same genomic DNA as that in Example 1 was used.

### 2. Example 2: Quality Examination 1

The quality of the DNA library obtained by the method presented in Example 1 was examined. First, in order to verify a bias toward a genomic region caused at the generation of a library, a comparative examination of the fragmented DNAs with the DNA library generated by the conventional technique was carried out as described in the above-mentioned Reference Example.

(Analysis Method)

Data of 850K reads per sample was acquired and analyzed on a personal computer equipped with Linux (registered trademark). Specifically, bowtie2 was used to map the genomic data. Next, the depth function of samtools was used to calculate the width of coverage for a reference genome in each sample.

(Results)

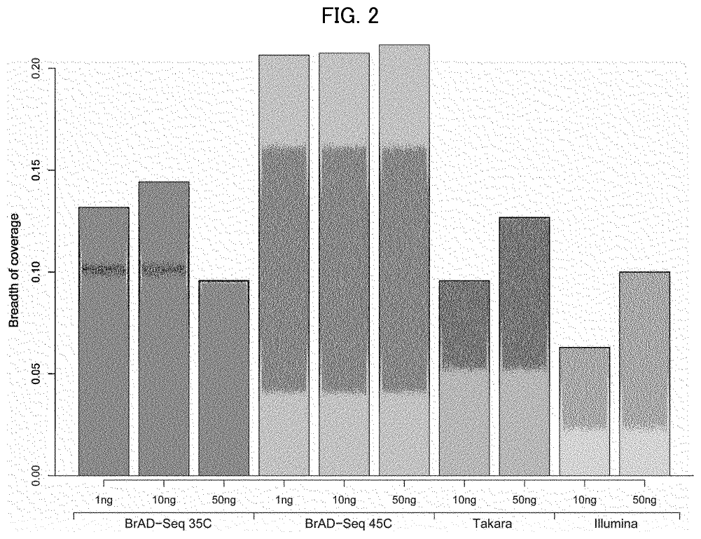

The results are shown in FIG. 2. FIG. 2 is a graph showing the ratio of sequenced genomic regions to the reference genome in each sample. In FIG. 2, the samples obtained in Example 1 are described as BrAD-Seq, and the samples obtained using the kits from Takara Bio Inc. and Illumina, Inc. are described as Takara and Illumina, respectively. The same applies to all graphs shown below.

Under the conditions of 45° C., higher values were obtained as compared to the values for the samples obtained using the kits from the other companies. This means that the genome was more widely mapped, and less bias was shown.

In terms of the number of reads, mapping rate, and GC content obtained from the sequencing, the method of the present invention could obtain the results similar to the results obtained by the kits from the other companies.

### 3. Example 3: Quality Examination 2

In order to further examine the quality of the DNA library obtained by the method presented in Example 1, the ratio of the read bases to the reference chromosome (chromosomes 1 to 5) in each sample obtained in Example and Reference Example was examined.

(Analysis Method)

From the result of the mapping in the above-mentioned [Example 2: Quality examination 1] section, the number of reads mapped to each chromosome was calculated using the idxstats function of samtools. Each read length was added to this value to calculate the total number of bases mapped to each chromosome. Finally, the total number of bases was divided by the total length of each chromosome, so that the coverage was calculated.

(Results)

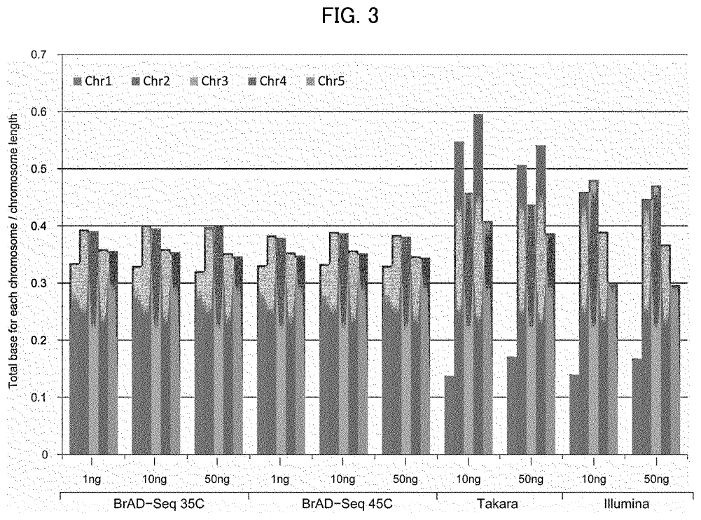

The results are shown in FIG. 3. FIG. 3 is a graph showing the ratio of read bases to the reference chromosome in each sample.

The present invention obtained a uniform value for each chromosome as compared with the existing kits. This means that the sequence information of each chromosome is obtained uniformly, and it can be said that there is less bias.

From the above, it was found that the DNA library obtained by the method of the present invention reflects the original genome length as compared with the existing techniques. It was also found that the method of the present invention is superior to the conventional methods at both 35° C. and 45° C.

### 4. Example 4: Quality Examination 3

Further, the mapping efficiency for the reference genome in each sample obtained in Example and Reference Example was determined.

(Analysis Method)

The result of the mapping in the above-mentioned [Example 2: Quality examination 1] section was converted into bed using the bamtobed, makewindows, and coverage functions of bedtools. Further, the mapping coverage per 1000 bp was calculated using bedtools.

(Results)

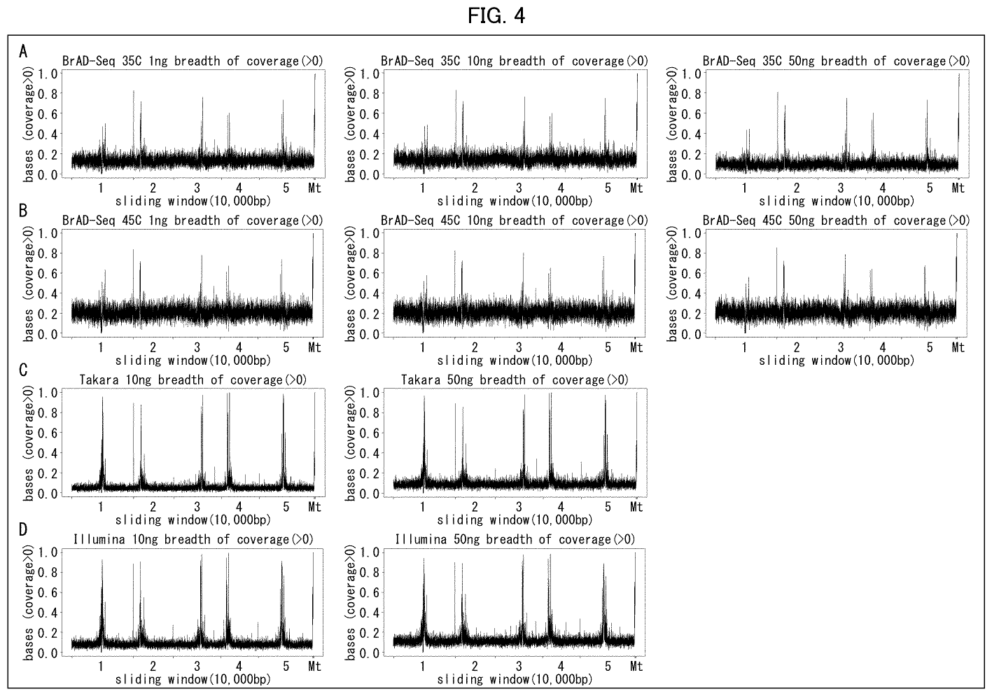

The results are shown in FIG. 4. FIG. 4 is a graph showing the mapping efficiency for the reference genome in each sample. A in FIG. 4 shows the results of BrAD-seq 35° C., B in FIG. 4 shows the results of BrAD-seq 45° C., C in FIG. 4 shows the results of Takara, and D in FIG. 4 shows the results of Illumina. Further, A and B in FIG. 4 are each data of the samples having 1 ng, 10 ng and 50 ng of input genomic DNA in the order from the left, and C and D in FIG. 4 are each data of the samples having 10 ng and 50 ng of input genomic DNA in the order from the left.

In a case where the existing kits were used, the baseline was low, and a high peak was found in a certain area. Comparatively, in the present invention, the baseline was high and wide, and uniform mapping was provided.

[5. Example 5: Generation of DNA library] Experimental material: 10 ng of genomic DNA of Drosophila melanogaster was used as an input.

Experimental method: The experiment was carried out by a procedure as indicated by the steps (a) to (d) below.

(Step (a): Acquisition of Fragmented dsDNA from Genomic DNA)

Fragmented dsDNA was obtained from genomic DNA under the following conditions.

Conditions for fragmentation of genomic DNA:
Duty factor 10%
Peak Incident power (w) 140
cycle/Burst 200
Time 80 sec
Model of Covaris used: 5220 (Step (b): Denaturation from dsDNA to ssDNA and Priming of 3′ End of ssDNA)

For the dsDNA obtained in the step (a) above, denaturation to ssDNA and priming of a 3′ end of the ssDNA were carried out by a procedure as indicated by 1) to 8) below. For the fragmented DNA sample with each concentration, three different annealing temperatures of 40° C., 45° C., and 50° C. were set.
1) The following materials were mixed.
5 μl of fragmented DNA (2 ng/μl)
1 μl of 3-prime priming adapter (SEQ ID NO: 1: 5′-GTGACTGGAGTTCAGACGTGTGCTCTTCCGATCTNNNNNNNN-3′) (5 μM L-3ILL-N8.2)
1.5 μl of 10× Buffer (500 mM Tris-HCl (pH 7.5 at 25° C.), 100 mM MgCl₂, 10 mM DTT) (Takara Bio Inc., RR006)
1.2 μl of dNTPs
6.225 μl of H₂O
0.075 μl of Ex taqcc
   15 μl in total
2) Incubation was carried out in a thermal cycler running the following program: 94° C. for 2 minutes; 40° C., 45° C., or 50° C. for 1 minute, 5 minutes, 10 minutes, or 15 minutes; 42° C. for 10 minutes; 72° C. for 5 minutes; and holding at 4° C.
3) 5 μl of 50 mM EDTA was added.
4) 30 μl of Ampure XP beads (Beckman Caulter) was added and mixed for size selection.
5) The resultant mixture was left to stand for 5 minutes.
6) The supernatant was discarded.
7) The remainder was washed twice with 200 μl of 80% EtOH.
8) The beads were dried.

(Step (c): Breath Capture of 5′ End of ssDNA (Breath Capturing))

Next, breath capture of a 5′ end of the ssDNA obtained in the step (b) above was carried out by a procedure as indicated by 1) to 8) below.
1) 4 μl of 10 μM 5-prime double stranded adapter oligo (5pSense8n (SEQ ID NO: 2: 5′-CCTACACGACGCTCTTCCGATCTNNNNNNNN-3′) and 5pAnti (SEQ ID NO: 3: 5′-AGATCGGAAGAGCGTCGTGTAGG-3′) was added.

2) The following mixtures were added.
1 μl of 10× Buffer (500 mM Tris-HCl (pH 7.5 at 25° C.), 100 mM MgCl₂, 10 mM DTT)
0.25 μl of 25 mM dNTPs
0.25 μl of DNA Pol I (Thermo Fisher Scientific, EP0041)
4.5 μl of H₂O
   6 μl in total
3) Incubation was carried out in a thermal cycler running the following program: 25° C. for 15 minutes.
4) The following mixtures were added.
10 μl of 50 mM EDTA
30 μl of ABR
   40 μl in total
4) The resultant mixture was left to stand for 5 minutes.
5) The supernatant was discarded.
6) The remainder was washed twice with 200 μl of 80% EtOH.
7) The beads were dried.
8) Elution was carried out with 30 μl of 10 mM Tris.

(Step (d): Concentration and Addition of Adapter Sequences)

Subsequently, the DNA eluted in the step (c) above was concentrated, and the adapter sequences were added by a procedure as indicated by 1) to 5) below.
1) The following PCR mixture was prepared:
2 μl of 10× Buffer (500 mM Tris-HCl (pH 7.5 at 25° C.), 100 mM MgCl₂, 10 mM DTT)
1.6 μl of dNTPs
1 μl of 2 μM PE1 (SEQ ID NO: 4: 5′-AATGATACGGCGACCACCGAGATCTACACTCTTTCCCTACACGA CGCTCTTCCGATCT-3′)
1 μl of 2 μM PE2 (SEQ ID NO: 5: 5′-CAAGCAGAAGACGGCATACGAGAT-index 8nt-GTGACTGGAGTTCAGACGTGTGCTCTTCCGAT-3′) (use a different index for each sample)
4.3 μl of H₂O
0.1 μl of Ex Taq
10 μl of breath captured DNA
20 μl in total
2) Incubation was carried out in a thermal cycler running the following program:
94° C. for 2 minutes; 18 cycles×(94° C. for 30 seconds; 65° C. for 30 seconds; 72° C. for 30 seconds); 72° C. for 7 minutes; and holding at 4° C.
3) Ampure XP beads purification was carried out with use of 0.8× beads (washing was carried out twice).
4) Elution was carried out with 10 μl of 10 mM Tris.
5) Sequencing was carried out by NovaSeq (Illumina, Inc.).

### 6. Example 6: Quality Examination

The quality of the DNA library obtained by the method presented in Example 4 was examined. Coverage to the genome was calculated to verify the effect of the annealing temperature.

(Analysis Method)

Data of 9M reads per sample was acquired and analyzed on a personal computer equipped with Linux (registered trademark). Specifically, bowtie2 was used to map the genomic data. Next, the depth function of samtools was used to calculate the width of coverage for the reference genome in each sample.

(Results)

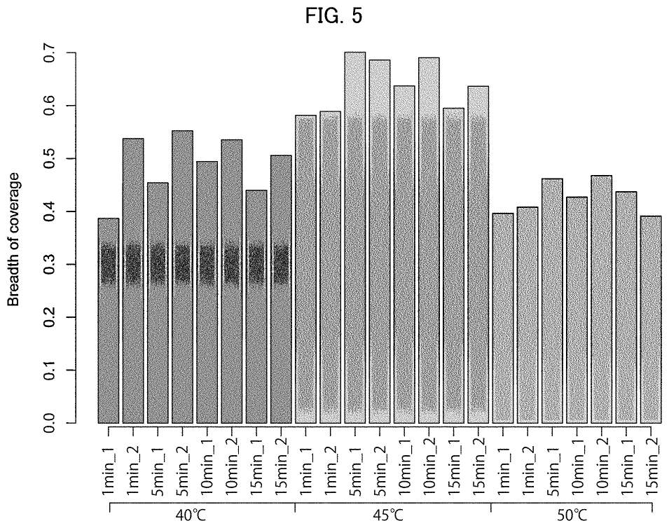

The results are shown in FIG. 5. FIG. 5 is a graph showing the ratio of sequenced genomic regions to the reference genome in each sample. In FIG. 5, the annealing temperature is shown at the bottom.

Under the reaction conditions of 45° C. for 5 minutes or 10 minutes, the highest values were obtained. This means that the genome was more widely mapped, and less bias was shown.

The result was that the method of the present invention presents no problem for genomic DNA derived from experimental animals, in terms of the number of reads, mapping rate, and GC content obtained from the sequencing.

### INDUSTRIAL APPLICABILITY

The present invention is applicable to, for example, the preparation of a DNA library used for next-generation genome sequencing (NGS) technologies and the like.Error! Bookmark not defined.

1. A method for producing a DNA molecule having adapter sequences added thereto, the method comprising:
a preparation step of preparing a double-stranded DNA in which a first DNA strand and a second DNA strand are at least partially hybridized; and
an annealing step of annealing a partially double-stranded oligonucleotide adapter to a 3′ end of the first DNA strand of the double-stranded DNA,
the partially double-stranded oligonucleotide adapter including a 3′ overhang which is a protruding end that is to be annealed to the 3′ end of the first DNA strand and that includes an oligonucleotide consisting of a random base sequence of at least 8 consecutive bases or a predetermined base sequence.

2. The method according to claim 1, wherein
a 5′ end of the first DNA strand, which constitutes the double-stranded DNA, includes a second adapter sequence having a base sequence that differs from each of base sequences of a first adapter sequence which is a double-stranded portion of the partially double-stranded oligonucleotide adapter.

3. The method according to claim 2, wherein
the preparation step includes preparing the double-stranded DNA by annealing, to a single-stranded DNA fragment corresponding to the second DNA strand, an adapter including: the oligonucleotide consisting of the random base sequence of the at least 8 consecutive bases or the predetermined base sequence; and the second adapter sequence located at a 5′ end of the oligonucleotide, and then extending a strand.

4. The method according to claim 3, wherein
the adapter is annealed to the single-stranded DNA fragment corresponding to the second DNA strand at a temperature in a range of not lower than 30° C. and not higher than 50° C.

5. The method according to claim 1, wherein
the single-stranded DNA fragment corresponding to the second DNA strand is a collection of a plurality of DNA fragments obtained by fragmenting a genomic DNA and denaturing the fragmented genomic DNA into single-stranded DNAs.

6. The method according to claim 1, wherein
said method includes extending a strand from the protruding end included in the partially double-stranded oligonucleotide adapter to generate a third DNA strand complementary to the first DNA strand.

7. The method according to claim 1, further comprising:
an amplification step of amplifying the double-stranded DNA in which the first DNA strand and a third DNA strand complementary to the first DNA strand are hybridized.

8. The method according to claim 7, wherein
a size of an obtained amplified fragment is in a range of not less than 300 bp and not more than 1000 bp.

9. A DNA library for next-generation sequencer analysis obtained by the method according to claim 7, said DNA library comprising a double-stranded DNA for analysis that is provided between at least a portion of the second adapter sequence and a sequence complementary to the second adapter sequence and at least a portion of the first adapter sequence which is a double-stranded portion of the partially double-stranded oligonucleotide adapter.

10. A kit for use in the method according to claim 1, said kit comprising at least one of the following (A) to (C):
(A) a partially double-stranded oligonucleotide adapter including a 3′ overhang which is a protruding end that is to be annealed to a 3′ end of a DNA strand and that includes an oligonucleotide consisting of a random base sequence of at least 8 consecutive bases or a predetermined base sequence;
(B) an adapter including: an oligonucleotide consisting of a random base sequence of at least 8 consecutive bases or a predetermined base sequence; and a second adapter sequence located at a 5′ end of the oligonucleotide; and
(C) a primer set consisting of a PCR primer that is to be annealed to a complementary sequence of the second adapter sequence and a PCR primer that is to be annealed to a block strand, which is a strand that does not have the protruding end, of the partially double-stranded oligonucleotide adapter.


SEQUENCE LISTING (5 sequences)

SEQ ID NO: 1 - DNA, 42 bp, Artificial Sequence
  3-prime priming adapter
  misc_feature (35)..(42): n is a, c, g, or t
gtgactggagttcagacgtgtgctcttccgatctnnnnnnnn

SEQ ID NO: 2 - DNA, 31 bp, Artificial Sequence
  5-prime double stranded adapter oligo 5pSense8n
  misc_feature (24)..(31): n is a, c, g, or t
cctacacgacgctcttccgatctnnnnnnnn

SEQ ID NO: 3 - DNA, 23 bp, Artificial Sequence
  5-prime double stranded adapter oligo 5pAnti
agatcggaagagcgtcgtgtagg

SEQ ID NO: 4 - DNA, 58 bp, Artificial Sequence
  PE1
aatgatacggcgaccaccgagatctacactctttccctacacgacgctcttccgatct

SEQ ID NO: 5 - DNA, 64 bp, Artificial Sequence
  PE2
  misc_feature (25)..(32): n is a, c, g, or t
caagcagaagacggcatacgagatnnnnnnnngtgactggagttcagacgtgtgctcttccgat